(19) United States
(12) Patent Application Publication
(10) Pub. No.: US 2023/0293618 A1
(43) Pub. Date: Sep. 21, 2023

(54) COMPOSITIONS COMPRISING ORANGE PEEL DERIVATIVES AND DICALCIUM PHOSPHATE AND METHODS OF USING

(71) Applicant: Jessica Karns, Sulphur Rock, AR (US)

(72) Inventor: Jessica Karns, Sulphur Rock, AR (US)

(21) Appl. No.: 18/109,162

(22) Filed: Feb. 13, 2023

Related U.S. Application Data
(60) Provisional application No. 63/309,489, filed on Feb. 11, 2022.

(51) Int. Cl. A61K 36/752; A61K 33/42; A61K 35/64; A61K 36/11; A61K 36/31; A61K 47/46; A61K 9/00; A61K 8/92; A61K 8/24; A61Q 11/00; A61P 19/02

(52) U.S. Cl. CPC A61K 36/752; A61K 8/24; A61K 8/922; A61K 9/006; A61K 33/42; A61K 35/64; A61K 36/11; A61K 36/31; A61K 47/46; A61P 19/02; A61Q 11/00

(57) ABSTRACT

Disclosed herein are compositions comprising a citrus peel composition, a phosphate composition, or a combination of both. The compositions disclosed herein may additionally comprise bee pollen, horsetail herb, cabbage powder, curcumin, epigallocatechin gallate, resveratrol, pterostilbene, niacin, NAD+ precursors, L-theanine, coenzyme Q10, coenzyme Q9, omega-3-fatty acids, phytophenols, flavonoids, ascorbic acid, folate, and stevia. The description herein describes methods for synergistically treating or ameliorating scoliosis, among other conditions or diseases. The compositions described herein may be formulated for oral, sublingual, dental, or intragingival delivery.

### CROSS REFERENCE

The present application for patent claims benefit of U.S. Provisional Pat. Application No. 63/309,489 filed Feb. 11, 2022, which is hereby incorporated by reference in its entirety.

### BACKGROUND

All citrus fruits belong to the family Rutacae. This family includes well-known examples such as lemons (Citrus limon), limes (Citrus aurantifolia), oranges (Citrus sinensis), and grapefruits (Citrus paradisi). Industry and consumers alike favor whole fruit and its respective juice for consumption, however the tough and bitter rind (or peel) serves little function for these purposes and accordingly, a significant amount of peel is generated as waste. Orange and other citrus peels can be used to produce extracts, essential oils, waxes, etc. For example, orange peel can be incorporated into various forms of oral care compositions, cosmetic agents, dietary supplements, texture or aroma enhancers, and flavoring agents.

Orange and other citrus peels are nutrient-dense and contain compounds that confer significant benefits, for example, flavonoids (polyphenols), vitamins, oils, minerals, phytophenols and other phytochemicals. Phytophenols can be used for their antioxidant effects and free radical scavenging abilities and can assist in the treatment of free radical diseases and ailments such as the effects of aging, cardiovascular dysfunction, and neurodegenerative diseases. Phytophenols can also support general health by deactivating carcinogenes and assisting in DNA repair. Flavonoids can include glycosides and flavones. Hesperidin and naringin are glycosides, while nobiletin and tangeretin are polymethoxylated flavones (PMF’s). Flavonoids can be utilized in compositions described herein for anticancer, antiviral, and anti-inflammatory activity.

Dicalcium phosphate, in addition to being an abrasive compound, can supply its subsequent ions to the body, calcium and phosphate. Calcium plays a role in forming new bone, clotting blood, nerve signaling, muscle contraction, and the release of hormones. Phosphate and the phosphorylation of compounds like glucose play an integral role in many metabolic processes. Glucose-6-phosphate, or G6P, is a phosphorylated form of glucose that serves as an important intermediary hub in glucose metabolism. G6P can feed into glycolysis, which anaerobically produces ATP in the cell’s cytoplasm. Glycolysis also catabolically produces pyruvate from G6P, which feeds into the citric acid cycle and cellular respiration in the mitochondria to further produce ATP. G6P is also used in the formation of glycogen, which is the stored form of glucose in the liver.

Without being bound by a particular theory, dicalcium phosphate is thought to increase cytoplasmic concentrations of phosphate, allowing for a faster turnover of ADP to ATP. ATP is used to fuel the formation of G6P from glucose. By increasing available levels of ATP and subsequently G6P, glycolysis can feed pyruvate to the mitochondria and increase its metabolic output. The mitochondria are responsible for producing substrates that methylate nuclear DNA. Nuclear DNA methylation, which helps regulate gene expression, is known to decrease with age. The hypomethylation of DNA seen with age is known to be associated with neurodegenerative and autoimmune diseases and cancer. Dicalcium phosphate may help to increase available calcium and phosphate levels, increase ATP and G6P concentrations, increase G6P production, increase nuclear DNA methylation, and treat neurodegenerative and autoimmune diseases.

While various benefits of both orange peel and dicalcium phosphate, individually, are known and/or have been theorized, the potentially synergistic effects of orange peel and dicalcium phosphate employed together have heretofore been unexplored.

### SUMMARY

Embodiments of the present disclosure relate to novel dietary supplements and oral care products comprising at least dicalcium phosphate and one or more orange peel derivatives, and various methods of using.

These and other features, aspects, and advantages of the present embodiments will become understood with reference to the following description, appended claims, and accompanying figures.

### BRIEF DESCRIPTION OF THE DRAWINGS

FIGS. 1A and 1B show x-rays illustrating a difference in cervical spine curvature and surrounding inflammation of a patient prior to administration of a composition according to the present disclosure (FIG. 1A) and approximately five months subsequent to administration of a composition according to the present disclosure (FIG. 1B).

FIGS. 2A and 2B show x-rays illustrating a difference in thoracic and lumbar spine curvature and surrounding inflammation of a patient prior to administration of a composition according to the present disclosure (FIG. 2A) and approximately three months subsequent to administration of a composition according to the present disclosure (FIG. 2B).

FIGS. 3A and 3B show x-rays illustrating a difference in thoracic and lumbar spine curvature and surrounding inflammation of a patient prior to administration of a composition according to the present disclosure (FIG. 3A) and approximately three months subsequent to administration of a composition according to the present disclosure (FIG. 3B).

FIGS. 4A and 4B show x-rays illustrating a difference in thoracic and lumbar spine curvature and surrounding inflammation of a patient prior to administration of a composition according to the present disclosure (FIG. 4A) and approximately eighteen months subsequent to administration of a composition according to the present disclosure (FIG. 4B).

FIGS. 5A and 5B show x-rays illustrating a difference in thoracic and lumbar spine curvature and surrounding inflammation of a patient prior to administration of a composition according to the present disclosure (FIG. 5A) and approximately eighteen months subsequent to administration of a composition according to the present disclosure (FIG. 5B).

### DETAILED DESCRIPTION

Some embodiments provide a composition comprising an amount of one or more citrus peel compounds formulated as a citrus peel composition. In certain embodiments, a citrus peel compound can comprise an amount of orange peel, an extract thereof, or a derivative thereof, lemon peel, an extract thereof, or a derivative thereof, lime peel, an extract thereof, or a derivative thereof, grapefruit peel, an extract thereof, or a derivative thereof, or any combination thereof. In some embodiments, a citrus peel composition, as disclosed herein, may comprise a pharmaceutically acceptable vehicle, carrier, or diluent. Some embodiments can be formulated to have varying amounts of these constituents.

When provided in a citrus peel composition, the amount of citrus peel compound provided may be about 10 µg to about 10 g. For example, the amount of a citrus peel compound in the citrus peel composition can be about 10 µg, 15 µg, 20 µg, 25 µg, 30 µg, 35 µg, 40 µg, 45 µg, 50 µg, 55 µg, 60 µg, 65 µg, 70 µg, 75 µg, 80 µg, 85 µg, 90 µg, 95 µg, 100 µg, 125 µg, 150 µg, 175 µg, 200 µg, 225 µg, 250 µg, 275 µg, 300 µg, 325 µg, 350 µg, 375 µg, 400 µg, 425 µg, 450 µg, 475 µg, 500 µg, 525 µg, 550 µg, 575 µg, 600 µg, 625 µg, 650 µg, 675 µg, 700 µg, 725 µg, 750 µg, 775 µg, 800 µg, 825 µg, 850 µg, 875 µg, 900 µg, 925 µg, 950 µg, 975 µg, 1000 µg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1.0 g, 1.25 g, 1.50 g, 1.75 g, 2.0 g, 2.25 g, 2.50 g, 2.75 g, 3.0 g, 3.25 g, 3.50 g, 3.75 g, 4.0 g, 4.25 g, 4.50 g, 4.75 g, 5.0 g, 5.25 g, 5.50 g, 5.75 g, 6.0 g, 6.25 g, 6.50 g, 6.75 g, 7.0 g, 7.25 g, 7.50 g, 7.75 g, 8.0 g, 8.25 g, 8.50 g, 8.75 g, 9.0 g, 9.25 g, 9.50 g, 9.75 g, 10.0 g, or any range or amount in between any two of the preceding values and any other ranges or amounts disclosed herein.

Some embodiments provide a composition comprising an amount of dicalcium phosphate formulated as a phosphate composition. In some embodiments, a phosphate composition, as disclosed herein, may comprise a pharmaceutically acceptable vehicle, carrier, or diluent. Some embodiments can be formulated to have varying amounts of these constituents.

When provided in a phosphate composition, the amount of dicalcium phosphate provided may be about 10 µg to about 10 g. For example, the amount of dicalcium phosphate provided in the phosphate composition can be about 10 µg, 15 µg, 20 µg, 25 µg, 30 µg, 35 µg, 40 µg, 45 µg, 50 µg, 55 µg, 60 µg, 65 µg, 70 µg, 75 µg, 80 µg, 85 µg, 90 µg, 95 µg, 100 µg, 125 µg, 150 µg, 175 µg, 200 µg, 225 µg, 250 µg, 275 µg, 300 µg, 325 µg, 350 µg, 375 µg, 400 µg, 425 µg, 450 µg, 475 ng, 500 µg, 525 µg, 550 µg, 575 µg, 600 µg, 625 µg, 650 µg, 675 µg, 700 µg, 725 µg, 750 µg, 775 µg, 800 µg, 825 µg, 850 µg, 875 µg, 900 µg, 925 µg, 950 µg, 975 µg, 1000 µg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1.0 g, 1.25 g, 1.50 g, 1.75 g, 2.0 g, 2.25 g, 2.50 g, 2.75 g, 3.0 g, 3.25 g, 3.50 g, 3.75 g, 4.0 g, 4.25 g, 4.50 g, 4.75 g, 5.0 g, 5.25 g, 5.50 g, 5.75 g, 6.0 g, 6.25 g, 6.50 g, 6.75 g, 7.0 g, 7.25 g, 7.50 g, 7.75 g, 8.0 g, 8.25 g, 8.50 g, 8.75 g, 9.0 g, 9.25 g, 9.50 g, 9.75 g, 10.0 g, or any range or amount in between any two of the preceding values and any other ranges or amounts disclosed herein.

Certain embodiments provide a combination composition comprising an amount of a citrus peel composition, as described herein, and an amount of a phosphate composition, as described herein, wherein the amount of a citrus peel composition and the amount of a phosphate composition are present in a ratio. In some embodiments, the ratio of the amount of a citrus peel composition to the amount of a phosphate composition may be about 1:1. In certain embodiments, the ratio of the amount of a citrus peel composition to the amount of a phosphate composition can be within the range of about 10:1 to about 1:10. In this regard, the ratio of the amount of a citrus peel composition to the amount of a phosphate composition may be about 10:1, 9.5:1, 9:1, 8.5:1, 8:1, 7.5:1, 7:1, 6.5:1, 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, or any ratio therebetween.

In some embodiments, the ratio of the amount of a citrus peel composition and the amount of a phosphate composition present in a combination composition, as described herein, may be a synergistic ratio. As used herein, a “synergistic ratio” refers to a ratio that elicits an unexpectedly superior pharmacological, physiological, nutritional, or nutraceutical effect in a subject. In some embodiments, the synergistic ratio of the amount of a citrus peel composition to the amount of a phosphate composition may be about 1:1. In certain embodiments, the synergistic ratio of the amount of a citrus peel composition to the amount of a phosphate composition can be within the range of about 5:1 to about 1:5. In this regard, the synergistic ratio of the amount of a citrus peel composition to the amount of a phosphate composition may be about 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, or any ratio therebetween. As used herein, an “unexpectedly superior effect” refers to an effect that is at least equal to the combined effects achieved by administration of either component alone, or more preferably, an effect that is greater than the combined effects achieved by administration of either component alone.

In some embodiments, a combination composition comprising a synergistic ratio, as described herein, may elicit unexpectedly superior effects in increasing cytosolic concentrations and/or production of G6P in a subject.

In certain embodiments, a combination composition comprising a synergistic ratio, as described herein, may elicit unexpectedly superior effects in treating and/or ameliorating a condition associated with the spine, such as scoliosis. In certain embodiments, treating/ameliorating scoliosis can comprise improving skeletal alignment, improving spinal alignments, improving bone health, improving bone density, and/or improving cranial alignment. In some embodiments, the superior effects may be achieved by administering the combination composition alone. In other embodiments, the aforementioned effects may further be improved by combining the composition with another therapy, such as chiropractic therapy or physical therapy.

In certain embodiments, a combination composition comprising a synergistic ratio, as described herein, may elicit unexpectedly superior effects in improving a subject’s range of motion.

In other embodiments, a combination composition comprising a synergistic ratio, as described herein, may elicit unexpectedly superior effects in increasing DNA methylation levels.

In some embodiments, a combination composition comprising a synergistic ratio, as described herein, may elicit unexpectedly superior effects in increasing folate levels in the brain of a subject.

In certain embodiments, a combination composition comprising a synergistic ratio, as described herein, may elicit unexpectedly superior effects in improving nerve impulse in a subject.

In certain embodiments, a combination composition comprising a synergistic ratio, as described herein, may elicit unexpectedly superior effects in treating and/or ameliorating cancer, more preferably breast cancer, or symptoms associated therewith.

In some embodiments, a combination composition comprising a synergistic ratio, as described herein, may elicit unexpectedly superior effects in treating and/or ameliorating cardiovascular disease or symptoms associated therewith.

In other embodiments, a combination composition comprising a synergistic ratio, as described herein, may elicit unexpectedly superior effects in reducing pain and/or inflammation. In some embodiments, the pain or inflammation being reduced is related to or results from a bone injury in a subject.

In certain embodiments, a combination composition comprising a synergistic ratio, as described herein, may elicit unexpectedly superior effects in treating and/or ameliorating autism spectrum disorder or symptoms associated therewith.

In some embodiments, a combination composition comprising a synergistic ratio, as described herein, may elicit unexpectedly superior effects in treating and/or ameliorating degenerative disk disease or symptoms associated therewith.

In certain embodiments, a combination composition comprising a synergistic ratio, as described herein, may elicit unexpectedly superior effects in treating and/or ameliorating the effects of neurodegenerative diseases/disorders, such as Alzheimer’s disease, Parkinson’s disease, dementia, multiple sclerosis, Huntington’s disease, motor neuron disease, and progressive supranuclear palsy, or any symptoms associated therewith.

In other embodiments, a combination composition comprising a synergistic ratio, as described herein, may elicit unexpectedly superior effects in treating and/or ameliorating osteoporosis or symptoms associated therewith.

In some embodiments, a combination composition comprising a synergistic ratio, as described herein, may elicit unexpectedly superior effects in improving low and/or poor metabolism.

In certain embodiments, a combination composition comprising a synergistic ratio, as described herein, may elicit unexpectedly superior effects in treating and/or ameliorating anxiety and/or depression or symptoms associated therewith.

In other embodiments, a combination composition comprising a synergistic ratio, as described herein, may elicit unexpectedly superior effects in treating and/or ameliorating muscular dystrophy or symptoms associated therewith.

In certain embodiments, a combination composition comprising a synergistic ratio, as described herein, may elicit unexpectedly superior effects in treating and/or ameliorating a fungal infection or symptoms associated therewith.

In other embodiments, a combination composition comprising a synergistic ratio, as described herein, may elicit unexpectedly superior effects in improving and/or stimulating immune function.

In some embodiments, a combination composition comprising a synergistic ratio, as described herein, may elicit unexpectedly superior effects in improving hearing and/or eyesight of a subject.

In certain embodiments, a combination composition comprising a synergistic ratio, as described herein, may elicit unexpectedly superior effects in improving the production of collagen, L-carnitine, and noradrenaline, the regulation of carnitine and noradrenaline neurotransmission, and/or the release of catecholamine and acetylcholine.

In other embodiments, a combination composition comprising a synergistic ratio, as described herein, may elicit unexpectedly superior effects in regulating blood glucose, improving NRF-2 activation, improving lymphatic flow, increasing glutathione production, and/or boosting satiety.

While the combination composition comprising an amount of a citrus peel composition, as described herein, and an amount of a phosphate composition, as described herein, is preferably used to obtain the aforementioned unexpected results, one may also observe effective results when using either the citrus peel composition or phosphate composition, by itself.

In some embodiments, a citrus peel composition, as described herein, a phosphate composition, as described herein, or a combination composition, as described herein, can comprise an amount of one or more supplement ingredients. As used herein, the term “supplement ingredient” can refer to essential fatty acids such as linolenic acid and linoleic acid, and essential amino acids such as tryptophan, lysine, methionine, phenylalanine, threonine, valine, leucine, isoleucine, arginine, L-theanine, histidine, and n-acetyl cysteine. Also included within the meaning of supplement ingredients are vitamins such as retinol (vitamin A), thiamine (vitamin B1), riboflavin (vitamin B2), niacin (vitamin B3), pantothenic acid (vitamin B5), pyridoxine, pyridoxamine, or pyridoxal (vitamin B6), biotin (vitamin B7) or pharmaceutically acceptable salts thereof, folic acid (vitamin B9) or pharmaceutically acceptable salts thereof, cobalamin (vitamin B12), choline, ascorbic acid (vitamin C) or pharmaceutically acceptable salts thereof, ergocalciferol (vitamin D2), calciferol (vitamin D3), 22-dihydroergocalciferol (vitamin D4), sitocalciferol (vitamin D5), tocopherol (vitamin E), phylloquinone (vitamin K1), menaquinone (vitamin K2), menadione (vitamin K3), or any combination of the foregoing. Other vitamins not explicitly listed would readily be envisaged by those of skill in the art, in view of the disclosure contained herein. Supplement ingredients can further include dietary minerals such as, for example, chromium, bromine, cobalt, copper, fluorine, germanium, iodine, iron, magnesium, manganese, molybdenum, potassium, selenium, silicon, zinc, calcium, phosphorous, sodium, sulfur, and vanadium. Supplement ingredients can also comprise cranberry extract, turmeric, royal jelly, açaí berry, bee pollen, beet root, coral calcium, oyster shell, gotu kola, horsetail herb, Gingko biloba, lions mane mushroom, pomegranate, hibiscus flower, strawberry powder, dandelion root, celery powder, parsley powder, peppermint leaf, cinnamon bark powder, curcumin, epigallocatechin gallate, resveratrol, pterostilbene, nicotinamide riboside, NAD+ precursors (nicotinamide riboside/nicotinamide mononucleotide), Coenzyme Q9, Coenzyme Q10, omega-3-fatty acids, cabbage powder, stevioside, rebaudoside, and combinations thereof. Supplement ingredients can include nitrates such as citrulline nitrate, creatine nitrate, beta-alanine nitrate, and the like. Of these, the preferred supplement ingredients are horsetail herb, cabbage powder, curcumin, epigallocatechin gallate, resveratrol, pterostilbene, niacin, nicotinamide riboside, nicotinamide mononucleotide, L-theanine, Coenzyme Q9, Coenzyme Q10, omega-3-fatty acids, stevia, and any combination thereof.

Horsetail herb (Equisetum arvense) is believed to have antioxidant activity and stimulate osteoblasts. Adding horsetail herb to an embodiment of the composition described herein may help to boost antioxidant activity and assist in the healing of bone injuries.

Cabbage powder is believed to increase antioxidant activity, reduce inflammation, support cardiovascular health, and help treat osteoporosis. Adding cabbage powder to an embodiment of the composition described herein may assist in maintaining general health, reducing inflammation, increasing antioxidant activity, and healing bone injuries.

Curcumin is believed to increase antioxidant activity and decrease inflammation. Adding curcumin to an embodiment of the composition described herein may help to maintain general health, increase antioxidant activity, and reduce inflammation.

Resveratrol is a potent phenol found in grape skin and has been shown to reduce inflammation through regulation of inflammatory pathways and contribute to overall cellular health, giving it its anti-aging effects. Adding resveratrol to an embodiment of the composition described herein may help to maintain general health, reduce inflammation, and provide anti-ageing benefits.

Pterostilbene has been shown to have antioxidant and anti-inflammatory activity and adding pterostilbene to an embodiment of the composition described herein may assist in maintaining general health, increasing antioxidant activity, and reducing inflammation.

Niacin (Vitamin B-3) is an antioxidant and a component of the metabolically integral molecules NAD+ and NADP+. Niacin is also involved in cell signaling and DNA repair. Adding niacin to an embodiment of the composition described herein may help to maintain general health, boost antioxidant activity, and boost metabolism.

Nicotinamide riboside is structurally similar to niacin and acts as a precursor to NAD+. Adding nicotinamide riboside to an embodiment of the composition described herein may help to maintain general health, boost metabolism, and provide anti-ageing benefits.

Nicotinamide mononucleotide is structurally similar to niacin, and, like nicotinamide riboside, acts as a precursor to NAD+. Adding nicotinamide mononucleotide to an embodiment of the composition described herein may help to maintain general health, boost metabolism, and provide anti-aging benefits.

L-theanine is an amino acid found in black tea and some mushrooms and is structurally similar to L-glutamate. It is believed that adding L-theanine to embodiments of the composition described herein may help to reduce anxiety, aid in sleep, and/or improve general health and well-being.

Coenzyme Q10 can be used as an antioxidant, to help produce ATP, and modulate immune function. Adding coenzyme Q10 to an embodiment of the composition described herein may help slow the progression of neurodegenerative diseases, improve general health and well-being, boost metabolism, and increase antioxidant activity. Adding coenzyme Q9 to an embodiment of the composition described herein may help to boost antioxidant activity, improve general health and well-being, and help increase coenzyme Q10 levels in the body.

Omega-3 fatty acids are essential fats that the body cannot make on its own and have been shown to improve cardiovascular health, lower inflammation, and help produce important hormones. Adding omega-3 fatty acids to an embodiment of the composition described herein can help improve general health and well-being, improve cardiovascular health, and lower inflammation.

Compositions described herein can include one or more of the foregoing supplement ingredients, as would be understood by one of skill in the art.

When provided in a citrus peel composition, as described herein, a phosphate composition, as described herein, or a combination composition, as described herein, the amount of the one or more supplement ingredients may be about 10 µg, 15 µg, 20 µg, 25 µg, 30 µg, 35 µg, 40 µg, 45 µg, 50 µg, 55 µg, 60 µg, 65 µg, 70 µg, 75 µg, 80 µg, 85 µg, 90 µg, 95 µg, 100 µg, 125 µg, 150 µg, 175 µg, 200 µg, 225 µg, 250 µg, 275 µg, 300 µg, 325 µg, 350 µg, 375 µg, 400 µg, 425 µg, 450 µg, 475 µg, 500 µg, 525 µg, 550 µg, 575 µg, 600 µg, 625 µg, 650 µg, 675 µg, 700 µg, 725 µg, 750 µg, 775 µg, 800 µg, 825 µg, 850 µg, 875 µg, 900 µg, 925 µg, 950 µg, 975 µg, 1000 µg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1.0 g, or any range or amount in between.

In some embodiments, a citrus peel composition, as described herein, a phosphate composition, as described herein, or a combination composition, as described herein, may further comprise an amount of at least one excipient. The excipients are not particularly limited. Exemplary excipients include, but are not limited to: acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); alkalizing agents (ammonia solution, ammonium carbonate, diethanolamine, diisopropanolamine, potassium hydroxide, sodium bicarbonate, sodium borate, sodium carbonate, sodium hydroxide, trolamine); antifoaming agents (dimethicone, simethicone); antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); chelating agents (edetate disodium, ethylenediaminetetraacetic acid and salts, edetic acid); coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein); colorants (caramel, red, yellow, black or blends, ferric oxide); complexing agents (ethylenediaminetetraacetic acid and salts (EDTA), edetic acid, gentisic acid ethanolamide, oxyquinoline sulfate); desiccants (calcium chloride, calcium sulfate, silicon dioxide); emulsifying and/or solubilizing agents (acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax); filtering aids (powdered cellulose, purified siliceous earth); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); humectants (glycerol, hexylene glycol, sorbitol); plasticizers (castor oil, diacetylated monoglycerides, diethyl phthalate, glycerol, mono- and di-acetylated monoglycerides, propylene glycol, triacetin, triethyl citrate); polymers (cellulose acetate, alkyl celluloses, hydroxyalkyl, acrylic polymers and copolymers); solvents (acetone, alcohol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or thickening agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium 12, carrageenan, microcrystalline and carboxymethylcellulose sodium cellulose, dextrin, gelatin (Bloom strength 50-100), guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner’s sugar, syrup); surfactants (simethicone); tablet binders (acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner’s sugar); tablet disintegrants (alginic acid, microcrystalline cellulose, croscarmellose sodium, crospovidone, polacrilin potassium, sodium starch glycolate, starch, pregelatinized starch); tablet and/or capsule lubricants (calcium stearate, glyceryl behenate, magnesium stearate, light mineral oil, sodium stearyl fumarate, stearic acid, purified stearic acid, talc, hydrogenated vegetable oil, zinc stearate); tonicity agent (dextrose, glycerol, mannitol, potassium chloride, sodium chloride); vehicle: flavored and/or sweetened (aromatic elixir, compound benzaldehyde elixir, iso-alcoholic elixir, peppermint water, sorbitol solution, syrup, tolu balsam syrup); vehicle: oleaginous (almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane); vehicle: solid carrier (sugar spheres); vehicle: sterile (Bacteriostatic water for injection, bacteriostatic sodium chloride injection); water repelling agents (cyclomethicone, dimethicone, simethicone); and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20 cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol). This list is not meant to be exclusive, but instead merely representative of the classes of excipients and the particular excipients that may be used in a citrus peel composition, as described herein, a phosphate composition, as described herein, or a combination composition, as described herein.

The compounds comprising a citrus peel composition, as described herein, a phosphate composition, as described herein, or a combination composition, as described herein, may be an active agent present in a therapeutically effective amount. By way of example, a “therapeutically effective amount” and/or an “effective amount” of the compound disclosed herein can be (on a dosage weight per subject weight basis), for example, 0.1 µg/kg, 0.5 µg/kg, 1 µg/kg, 1.5 µg/kg, 2.0 µg/kg, 2.5 µg/kg, 3.0 µg/kg, 3.5 µg/kg, 4.0 µg/kg, 4.5 µg/kg, 5.0 µg/kg, 10 µg/kg, 15 µg/kg, 20 µg/kg, 25 µg/kg, 30 µg/kg, 35 µg/kg, 40 µg/kg, 45 µg/kg, 50 µg/kg, 55 µg/kg, 60 µg/kg, 65 µg/kg, 70 µg/kg, 75 µg/kg, 80 µg/kg, 85 µg/kg, 90 µg/kg, 95 µg/kg, 100 µg/kg, 150 µg/kg, 200 µg/kg, 250 µg/kg, 300 µg/kg, 350 µg/kg, 400 µg/kg, 450 µg/kg, 500 µg/kg, 550 µg/kg, 600 µg/kg, 650 µg/kg, 700 µg/kg, 750 µg/kg, 80 µg/kg 0, 850 µg/kg, 900 µg/kg, 1 mg/kg, 1.5 mg.kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, or more, or any fraction or integer in between any two of the preceding amounts of the compound. An effective amount may include any of the ranges and amounts discussed herein.

Accordingly, in some embodiments, the dose of the compositions disclosed herein (corresponding to the therapeutically effective amount), can be about 10 µg to about 10 g per day. For example, the amount of the composition can be 10 µg, 15 µg, 20 µg, 25 µg, 30 µg, 35 µg, 40 µg, 45 µg, 50 µg, 55 µg, 60 µg, 65 µg, 70 µg, 75 µg, 80 µg, 85 µg, 90 µg, 95 µg, 100 µg, 125 µg, 150 µg, 175 µg, 200 µg, 225 µg, 250 µg, 275 µg, 300 µg, 325 µg, 350 µg, 375 µg, 400 µg, 425 µg, 450 µg, 475 µg, 500 µg, 525 µg, 550 µg, 575 µg, 600 µg, 625 µg, 650 µg, 675 µg, 700 µg, 725 µg, 750 µg, 775 µg, 800 µg, 825 µg, 850 µg, 875 µg, 900 µg, 925 µg, 950 µg, 975 µg, 1000 µg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1.0 g, 1.25 g, 1.50 g, 1.75 g, 2.0 g, 2.25 g, 2.50 g, 2.75 g, 3.0 g, 3.25 g, 3.50 g, 3.75 g, 4.0 g, 4.25 g, 4.50 g, 4.75 g, 5.0 g, 5.25 g, 5.50 g, 5.75 g, 6.0 g, 6.25 g, 6.50 g, 6.75 g, 7.0 g, 7.25 g, 7.50 g, 7.75 g, 8.0 g, 8.25 g, 8.50 g, 8.75 g, 9.0 g, 9.25 g, 9.50 g, 9.75 g, 10.0 g, or more, or any range or amount in between any two of the preceding values and any other ranges or amounts disclosed herein.

In some embodiments, compositions, as described herein, can be administered via methods described elsewhere herein on an hourly basis, e.g., every one, two, three, four, five, six, seven, eight, nine, ten, eleven, twelve, thirteen, fourteen, fifteen, sixteen, seventeen, eighteen, nineteen, twenty, twenty-one, twenty-two, twenty-three hours, or any interval in between, or on a daily basis, every two days, every three days, every four days, every five days, every six days, every week, every eight days, every nine days, every ten days, every two weeks, every month, or more or less frequently, as needed to achieve the desired therapeutic effect.

In some embodiments, a ramping administration protocol, i.e., where a subject is administered temporally increasing amounts of compositions described herein, can be utilized. For example, a subject could be administered with 100 mg of a composition, as described herein, once per day for 7 days, followed by 200 mg per day for the next 7 days, followed by 300 mg per day for the next 7 days. Administration protocols can also follow a pattern whereby the dosage amount decreases over time. For example, 300 mg of a composition, as described herein, per day for 7 days, followed by 200 mg per day for the next 7 days, followed by 100 mg per day for the next 7 days. In some embodiments, the methods as described herein can be utilized in combination with a calorie restriction protocol in a subject. In certain embodiments, the compositions described herein may be administered before, after, or during a meal. In addition, the appropriate dosage of the compositions can depend, for example, on the condition to be treated, the severity and course of the condition, whether the composition is administered for preventive or therapeutic purposes, previous therapy, the patient’s clinical history and response to the composition, the type of composition used, and the discretion of the attending physician. The composition can be suitably administered to the patient at one time or over a series of treatments and may be administered to the patient at any time from diagnosis onwards. The composition may be administered as the sole treatment or in conjunction with other drugs or therapies useful in treating the condition in question.

In certain embodiments, a citrus peel composition, as described herein, a phosphate composition, as described herein, or a combination composition, as described herein, can be formulated as a dietary supplement, nutritional supplement, or pharmaceutical agent.

In some embodiments, a citrus peel composition, as described herein, a phosphate composition, as described herein, or a combination composition, as described herein, may be formulated to be administered via a route including, but not limited to buccally, dentally, interstitially, intragingivally, intramuscularly, intraperitoneally, intravenously, nasally, ophthalmically, orally, parenterally, rectally, subcutaneously, sublingually, or topically.

In certain embodiments, a citrus peel composition, as described herein, a phosphate composition, as described herein, or a combination composition, as described herein, formulated to be administered via an oral route or a sublingual route may be in the form of a powder, a granule, a suspension, an aqueous solution, an oil-based solution, a beverage, a mouthwash, a syrup, an elixir, an emulsion, a capsule (soft, hard, gel, or plant-based), a pill, a tablet, a caplet, a sachet, a gum, a paste, a gel, or a dissolvable oral strip. In the aforementioned oral or sublingual formulations, the formulation may comprise one or more of the aforementioned excipients. One skilled in the art would recognize the excipients appropriate for the particular oral or sublingual delivery system contemplated. In addition, various methods of time release and location specific release are contemplated, including immediate release, delayed release, sustained release, controlled release, and targeted delivery.

In some embodiments, when a citrus peel composition, as described herein, a phosphate composition, as described herein, or a combination composition, as described herein, is formulated for oral delivery system, such formulation may be intended to provide delivery of the active compounds via an intragingival or dental route. In such embodiments, a composition, as described herein, may be in the form of a paste or gel, more specifically a toothpaste or tooth gel. In the gingival or dental formulations, the formulation may comprise one or more of the aforementioned excipients. One skilled in the art would recognize the excipients appropriate for the particular gingival/dental delivery system contemplated. In addition, various methods of time release and location specific release are contemplated, including immediate release, delayed release, sustained release, and controlled release.

Compositions intended for oral use can be prepared according to any method known in the art for the manufacture of pharmaceutically acceptable compositions and such compositions may include one or more of the following agents: sweeteners, flavoring agents, coloring agents, coatings, and preservatives. The sweetening and flavoring agents will increase the palatability of the preparation. Tablets containing the complexes in admixture with non-toxic pharmaceutically acceptable excipients suitable for tablet manufacture are acceptable. Pharmaceutically acceptable vehicles such as excipients are compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin, or acacia; and lubricating agents such as magnesium stearate, stearic acid or talc. Tablets can be uncoated or can be coated by known techniques to delay disintegration and absorption in the gastrointestinal tract and thereby provide a sustained action over a longer period of time. For example, a time delay material such as glyceryl monostearate or glyceryl distearate alone or with a wax can be employed.

Formulations for oral use can also be presented as hard gelatin-containing or non-gelatinous capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin, or olive oil. Aqueous suspensions can contain the complex of the described herein admixed with excipients suitable for the manufacture of aqueous suspensions. Such excipients include suspending agents, dispersing, or wetting agents, one or more preservatives, one or more coloring agents, one or more flavoring agents and one or more sweetening agents such as sucrose or saccharin.

Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules suitable for preparation of an aqueous suspension by the addition of water can provide the active ingredient in admixture with a dispersing or wetting agent, a suspending agent, and one or more preservatives. Additional excipients, for example sweetening, flavoring, and coloring agents, can also be present.

Syrups and elixirs can be formulated with sweetening agents, such as glycerol, sorbitol, or sucrose. Such formulations can also contain a demulcent, a preservative, a flavoring, or a coloring agent.

It will be appreciated that the amount of the composition may be combined with a carrier material to produce a single dosage form. Such forms will vary depending upon the host treated and the particular mode of administration.

Aqueous suspensions may contain the compound disclosed herein in admixture with excipients suitable for the manufacture of aqueous suspensions. Such excipients include suspending agents, dispersing, or wetting agents, one or more preservatives, one or more coloring agents, one or more flavoring agents and one or more sweetening agents such as sucrose or saccharin.

Utilization of controlled release vehicles would readily be envisaged by those of skill in the pharmaceutical sciences in view of the disclosure contained herein, and these aspects can be applied to nutritional and dietary supplements.

Numerous controlled release vehicles can be used, including biodegradable or bioerodable polymers such as polylactic acid, polyglycolic acid, and regenerated collagen. Controlled release drug delivery devices can include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released, and can be used in the disclosed methods.

Controlled release preparations can be achieved by the use of polymers to form complexes with or absorb a composition, as described herein. The controlled delivery can be exercised by selecting appropriate macromolecules such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active complex.

Controlled release of compositions, as described herein, can be taken to mean any of the extended release dosage forms. The following terms may be considered to be substantially equivalent to controlled release for the purposes of the present disclosure: continuous release, controlled release, delayed release, depot, gradual release, long term release, programmed release, prolonged release, programmed release, proportionate release, protracted release, repository, retard, slow release, spaced release, sustained release, time coat, time release, delayed action, extended action, layered time action, long acting, prolonged action, sustained action medications and extended release, release in terms of pH level in the gut and intestine, breakdown of the molecule and based on the absorption and bioavailability.

Hydrogels, wherein a composition, as disclosed herein, is dissolved in an aqueous constituent to gradually release over time, can be prepared by copolymerization of hydrophilic mono-olefinic monomers, such as ethylene glycol methacrylate. Matrix devices, wherein a composition, as described herein, is dispersed in a matrix of carrier material, can be used. The carrier can be porous, non-porous, solid, semi-solid, permeable, or impermeable. Alternatively, a device comprising a central reservoir of a composition disclosed herein surrounded by a rate controlling membrane can be used to control the release of the composition. Rate controlling membranes include ethylene-vinyl acetate copolymer or butylene terephthalate/polytetramethylene ether terephthalate. Use of silicon rubber or ethylene-vinyl alcohol depots are also contemplated.

Controlled release oral formulations can also be used. In some embodiments, a composition, as described herein, can be incorporated into a soluble or erodible matrix, such as a pill or a lozenge. In another example, the oral formulations can be a liquid used for sublingual administration. These liquid compositions can also be in the form a gel or a paste. Hydrophilic gums, such as hydroxymethylcellulose, can be used. A lubricating agent such as magnesium stearate, stearic acid, or calcium stearate can be used to aid in the tableting process.

In some embodiments, dosing for oral administration may be with a regimen calling for single daily dose, or for a single dose every other day, or for a single dose within 72 hours of the first administered dose, or for multiple, spaced doses throughout the day. The active compositions, which make up the therapy, may be administered simultaneously, either in a combined dosage form or in separate dosage forms intended for substantially simultaneous oral administration. The active compositions, which make up the therapy may also be administered sequentially, with either active composition being administered by a regimen calling for two-step ingestion. Thus, a regimen may call for sequential administration of the active compositions with spaced-apart ingestion of the separate, active compositions. The time period between the multiple ingestion steps may range from a few minutes to as long as about 72 hours, depending upon the properties of each active composition, such as potency, solubility, bioavailability, plasma half-life and kinetic profile of the composition, as well as depending upon the age and condition of the patient. The active compositions of the therapy whether administered simultaneously, substantially simultaneously, or sequentially, may involve a regimen calling for administration of one active composition by sublingual route and the other active composition by dental/gingival route. In one aspect, the embodiments described herein can achieve therapeutic and/or nutraceutical benefits not previously recognized or achievable, and thus, unexpectedly, and surprisingly achieve improved abilities for using the compositions.

The term “pharmaceutically acceptable salts” includes salts of the active compounds, which are prepared with relatively nontoxic acids or bases, depending on the particular substituents found on the compounds comprising the compositions described herein. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-tolylsulfonic acid, citric acid, tartaric acid, methanesulfonic acid, and the like. Also included are salts of amino acids such as arginate and the like, and salts of organic acids like glucuronic or galacturonic acids, and the like (see, for example, Berge et al., Journal of Pharmaceutical Science, 66: 1-19 (1977), which is hereby incorporated by reference in its entirety). Certain specific compounds of the present application contain both basic and acidic functionalities that allow the compounds to be converted into either base or acid addition salts.

As used herein, the term “pharmaceutically acceptable solvent” can refer to water or aqueous buffer solutions that are physiologically compatible, or aqueous solutions containing organic solvents that are physiologically compatible. A non-comprehensive list of pharmaceutically acceptable solvents is provided in U.S. Department of Health & Human Services, Food & Drug Administration, “Guidance for Industry: Q3C Impurities: Residual Solvents,” December 1997 or its current issue.

The terms “dietary supplement,” or “nutritional supplement” have the meaning ascribed to them under the Federal Food, Drug & Cosmetic Act.

As used herein, the term “excipient” refers to any compound that is part of a formulation that is not an active ingredient, i.e., one that has no relevant biological activity, and which is added to the formulation to provide specific characteristics to the dosage form, including by way of example, providing protection to the active ingredient from chemical degradation, facilitating release of a tablet or caplet from equipment in which it is formed, and so forth.

As used herein, the term “extract” means that the referenced compound can be physically or chemically altered to produce one or more compounds capable of being incorporated into the compositions described herein. For example, an extract can be a non-natural compound that is chemically distinct from that which exists in nature by virtue of subjecting a compound or raw material to human-controlled manufacturing or processing techniques, such as those described herein. In certain instances, an extract can refer to a non-natural composition that has had undesired components removed, thereby producing a compound that has markedly different characteristics from that which exists in nature or that possesses an enlarged function compared to natural compositions. In some instances, a composition described herein can deviate from any natural composition by virtue of being formulated into a non-natural combination of constituents, such as those described herein.

The term “pharmaceutical formulation”, “formulation”, “composition,” and the like can refer to preparations that are in such a form as to permit the biological activity of the active ingredients to be effective, and therefore may be administered to a subject for therapeutic use along with pharmaceutical, dietary, and/or nutritional supplement use. The meaning of these terms will be clear to the skilled artisan based upon the context in which they are used.

A “therapeutically effective amount,” as used herein, includes within its meaning, a non-toxic but sufficient amount of a compound active ingredient or composition comprising the same for use in the embodiments disclosed herein to provide the desired therapeutic effect. Similarly, “an amount effective to” or “an effective amount,” as used herein, includes within its meaning, a non-toxic but sufficient amount of a compound active ingredient or composition comprising the same to provide the desired effect. A “therapeutically effective amount” or an “effective amount” includes amounts of compounds that would not be achievable through a standard or natural diet, but requires supplementation and dosing, as described herein, to achieve specific, non-natural outcomes as set forth herein, along with expanded utilization of any compounds originating from or derived from natural sources. The exact amount of the active ingredient required will vary from subject to subject depending on factors such as the species being treated, the age and general condition of the subject, the severity of the condition being treated, the particular composition being administered, the weight of the subject, the mode of administration, and so forth. Thus, it may not always be possible to specify an exact “effective amount.” However, for any given case, an appropriate “effective amount” may be determined by one of ordinary skill in the art in view of the disclosure contained herein.

As used herein, the term “bioavailability” refers to the amount of a substance that is absorbed by a subject and ultimately available for biological activity in a subject’s tissue and cells.

As used herein, “identifying,” refers to detecting or selecting a subject from a population of potential subjects, for example, to establish that a particular subject possesses certain properties or characteristics. “Identifying” may include, for example, self-identification, self-diagnosis, and diagnosis by a medical professional.

As used herein, the terms “preventing”, “treating”, “treatment,” “alleviating,” “ameliorating,” and the like are used herein to generally refer to obtaining a desired pharmacological, physiological, nutritional, and/or nutraceutical effects, the scopes and meanings of which will be clear to the skilled artisan based upon the context in which these terms are used. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment” as used herein encompasses any treatment of a disease in a mammal, particularly a human, and includes: (a) preventing the disease from occurring in a subject that may be predisposed to the disease but has not yet been diagnosed as having it; (b) inhibiting the disease or arresting its development; or (c) relieving the disease, causing regression of the disease and/or its symptoms, conditions, and co-morbidities. In some embodiments, a composition, as described herein, may be administered to maintain healthy levels of a certain condition or biomarker in a subject, such as for example maintaining a healthy level of mental acuity. As set forth herein, any composition that is administered to prevent, treat, alleviate, or ameliorate any condition, can also be administered to maintain a healthy level of a physiological or biological condition. In certain embodiments, a nutritional supplement is administered to maintain a healthy level of one or more of the conditions disclosed herein. The scope and meaning of “preventing,” “treating,” “treatment,” “alleviating,” “ameliorating,” and “maintaining healthy levels of” would be immediately envisaged by the skilled artisan when viewing the term in the context of the disclosure and the claims.

As provided herein, the disclosure of a “ratio” of compounds and compositions corresponds to a ratio provided in terms of mass of the components present in the ratio.

When used in this disclosure, the phrase “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and can or cannot be present depending upon whether or not they affect the activity or action of the listed elements. For example, the use of a composition “consisting essentially of a composition” for the treatment of a particular disease or disorder, or the maintenance of a healthy condition, would exclude other ingredients that would materially alter the intended outcome of the composition.

As used herein, a composition that “substantially” comprises a compound means that the composition contains more than about 80% by weight, more preferably more than about 90% by weight, even more preferably more than about 95% by weight, and most preferably more than about 98% by weight of the compound.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

In addition, the appropriate dosage of the compositions can depend, for example, on the condition to be treated, the severity and course of the condition, whether the composition is administered for preventive or therapeutic purposes, previous therapy, the patient’s clinical history and response to the composition, the type of composition used, and the discretion of the attending physician. The composition can be suitably administered to the patient at one time or over a series of treatments and may be administered to the patient at any time from diagnosis onwards. The composition may be administered as the sole treatment or in conjunction with other drugs or therapies useful in treating the condition in question.

The present disclosure provides citrus peel compositions, phosphate compositions, or a combination composition thereof as solid/liquid dosage forms. By providing such dosage forms the aforementioned compositions are present in an unnatural form, i.e., is presented in a supplement (e.g., in a sublingual tablet, oral paste, or oral gel), that is different from that which occurs naturally, or the nutritional or dietary supplement results in unnatural supplementation that is unachievable through a non-supplemented diet.

While exemplary embodiments, aspects and variations have been provided herein, those of skill in the art will recognize certain modifications, permutations, additions and combinations and certain sub-combinations of the embodiments, aspects, and variations. It is intended that the following claims are interpreted to include all such modifications, permutations, additions and combinations and certain sub-combinations of the embodiments, aspects and variations are within their scope.

### EXAMPLES

Below are exemplary regimens for administering various exemplary compositions comprising a synergistic combination of at least dicalcium phosphate and orange peel derivative in accordance with various embodiments of the present disclosure.

### Example 1

In this example, a subject will be administered a dosage comprising an effective amount of a synergistic combination of orange peel powder and dicalcium phosphate, and optionally, one or more of bee pollen, horsetail extract, cabbage powder, and stevia. Measurements will be taken to determine the straightness of the subject’s teeth prior to initiating the regimen. After beginning the regimen where the effective amount of the synergistic combination of orange peel powder and dicalcium phosphate, measurements will be taken at certain intervals to determine the straightness of the subject’s teeth.

### Example 2

In this example, a subject will be administered a dosage comprising an effective amount of a synergistic combination of orange peel powder and dicalcium phosphate, and optionally, one or more of bee pollen, horsetail extract, cabbage powder, and stevia. Measurements will be taken to determine the subject’s DNA methylation prior to initiating the regimen. After beginning the regimen where the effective amount of the synergistic combination of orange peel powder and dicalcium phosphate, measurements will be taken at certain intervals to determine the subject’s DNA methylation.

### Example 3

In this example, a subject exhibiting scoliosis was administered a dosage comprising an effective amount of a synergistic combination of orange peel powder and dicalcium phosphate, and bee pollen, horsetail extract, cabbage powder, and stevia. The composition contained 4 parts orange peel powder, 4 parts dicalcium phosphate, 4 parts bee pollen, 1 part horsetail extract, 1 part cabbage powder, and 1 part stevia (stevioside and rebaudoside). The composition was administered to a subject three times daily; twice as an oral toothpaste administered to the subject by brushing their teeth with about ⅛ to ¼ of a teaspoon of the composition, and once as a sublingual tablet.

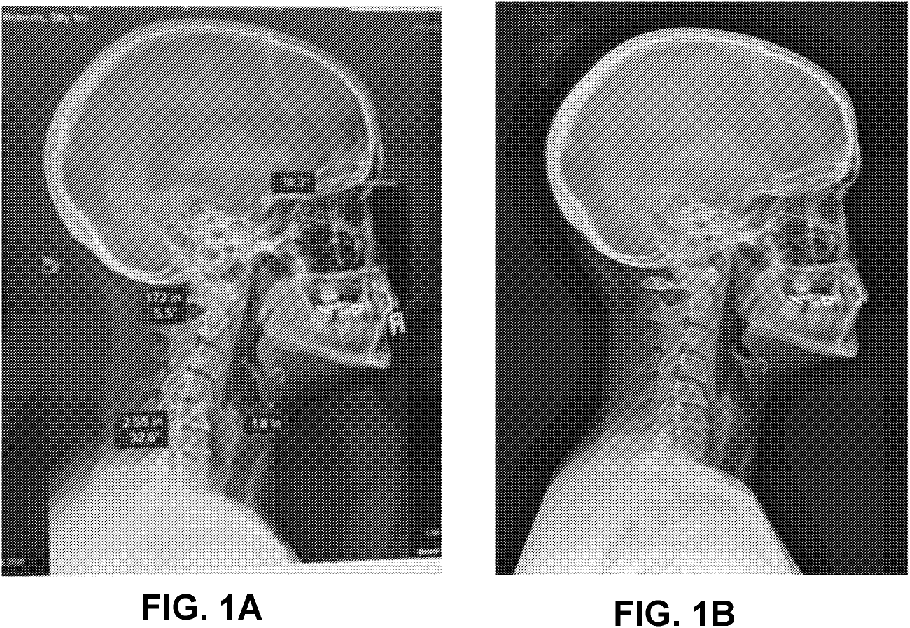

Measurements were taken to determine the condition of the subject’s spine prior to initiating the regimen. After beginning the regimen, measurements were taken at certain intervals to determine the condition of the subject’s spine. The results of which are shown in FIGS. 1-5.

FIGS. 1A and 1B show x-rays illustrating a difference in cervical spine curvature and surrounding inflammation of a patient prior to administration of a composition according to the present invention (FIG. 1A) and subsequent to administration of a composition according to the present invention (FIG. 1B). Although FIG. 1B does not quantify the illustrated curvature, the reduction in curvature is apparent, as is the reduction in inflammation. The interval between the taking the x-rays shown in FIGS. 1A and 1B was approximately five months.

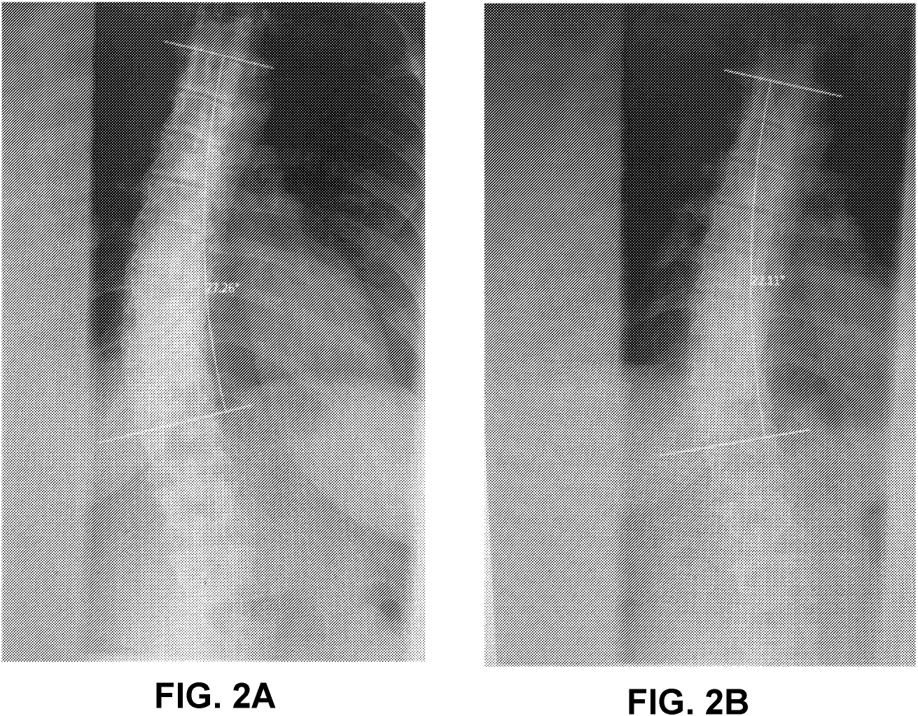

FIGS. 2A and 2B show x-rays illustrating a difference in thoracic and lumbar spine curvature and surrounding inflammation of a patient prior to administration of a composition according to the present invention (FIG. 2A) and subsequent to administration of a composition according to the present invention (FIG. 2B). As can be seen by comparing the quantification of curvature shown in FIG. 2A (27.25 degrees) with that shown in FIG. 2B (22.11 degrees), a reduction of 5.14 degrees was achieved, as was an apparent reduction in inflammation. The interval between the taking the x-rays shown in FIGS. 2A and 2B was approximately three months.

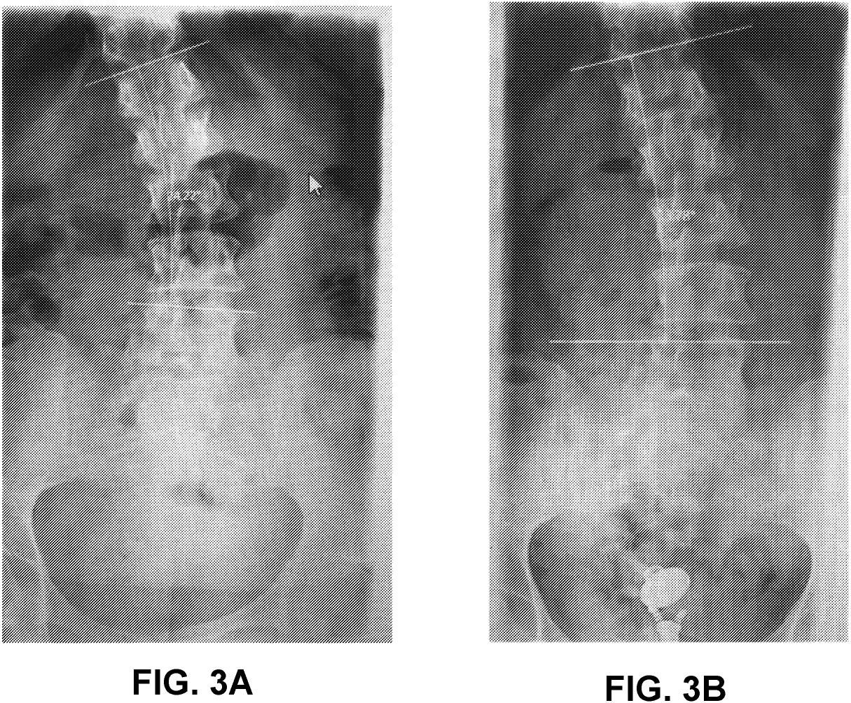

FIGS. 3A and 3B show x-rays illustrating a difference in thoracic and lumbar spine curvature and surrounding inflammation of a patient prior to administration of a composition according to the present invention (FIG. 3A) and subsequent to administration of a composition according to the present invention (FIG. 3B). As can be seen by comparing the quantification of curvature shown in FIG. 3A (24.22 degrees) with that shown in FIG. 3B (13.78 degrees), a reduction of 10.44 degrees was achieved, as was an apparent reduction in inflammation. The interval between the taking the x-rays shown in FIGS. 3A and 3B was approximately three months.

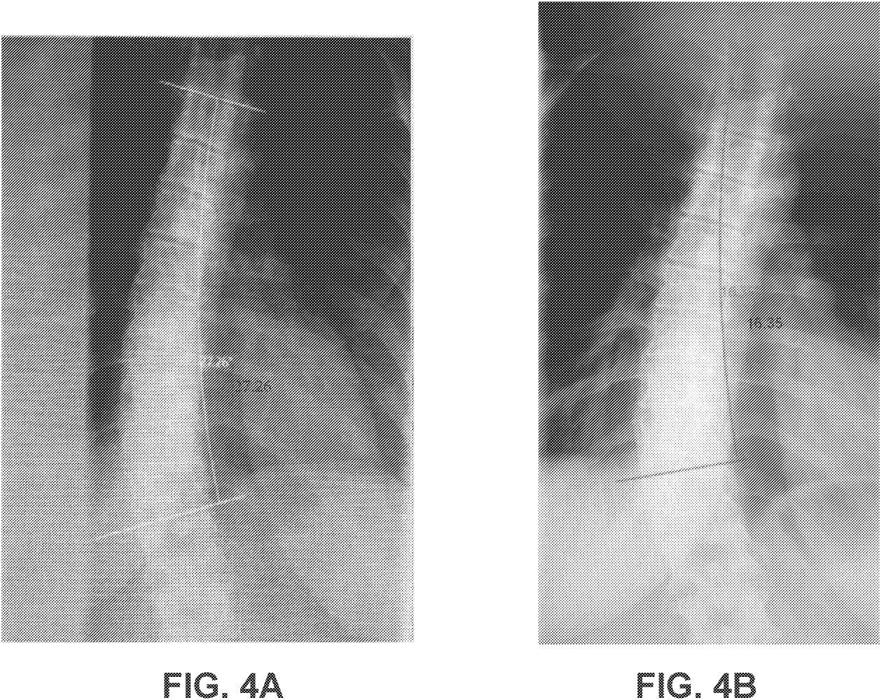

FIGS. 4A and 4B show x-rays illustrating a difference in thoracic and lumbar spine curvature and surrounding inflammation of a patient prior to administration of a composition according to the present invention (FIG. 4A) and subsequent to administration of a composition according to the present invention (FIG. 4B). As can be seen by comparing the quantification of curvature shown in FIG. 4A (27.26 degrees) with that shown in FIG. 4B (16.35 degrees), a reduction of 10.91 degrees was achieved, as was an apparent reduction in inflammation. The interval between the taking the x-rays shown in FIGS. 4A and 4B was approximately eighteen months.

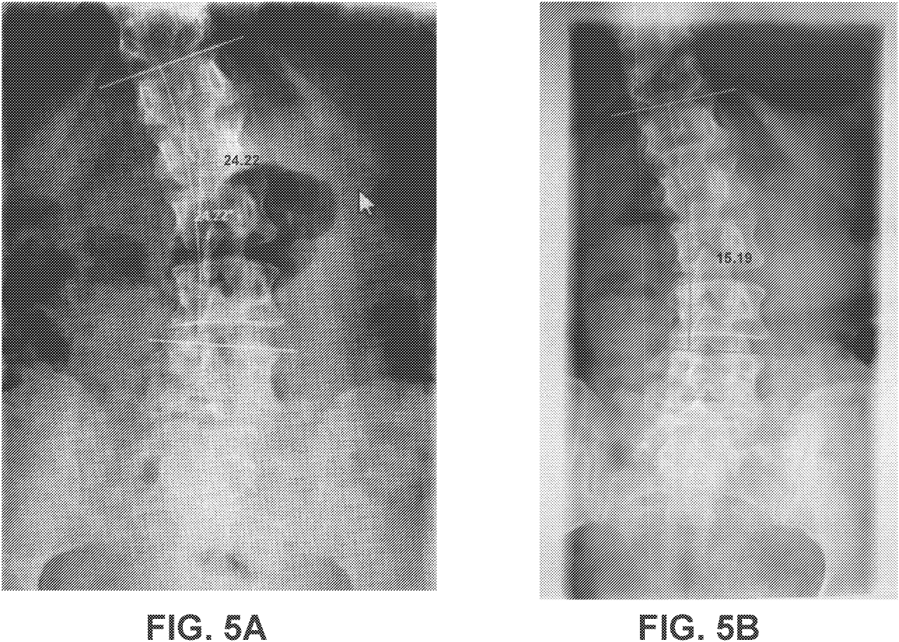

FIGS. 5A and 5B show x-rays illustrating a difference in thoracic and lumbar spine curvature and surrounding inflammation of a patient prior to administration of a composition according to the present invention (FIG. 5A) and subsequent to administration of a composition according to the present invention (FIG. 5B). As can be seen by comparing the quantification of curvature shown in FIG. 5A (24.22 degrees) with that shown in FIG. 5B (15.19 degrees), a reduction of 9.03 degrees was achieved, as was an apparent reduction in inflammation. The interval between the taking the x-rays shown in FIGS. 5A and 5B was approximately eighteen months.

### Example 4

In this example, a subject with breast cancer will be administered a dosage comprising an effective amount of a synergistic combination of orange peel powder and dicalcium phosphate, and optionally, one or more of bee pollen, horsetail extract, cabbage powder, and stevia. Measurements are taken to determine the condition of the subject’s breast cancer, including tumor size and malignancy prior to initiating the regimen. After beginning the regimen where the effective amount of the synergistic combination of orange peel powder and dicalcium phosphate, measurements will be taken at certain intervals to determine the condition of the subject’s breast cancer, including tumor size and malignancy.

What is claimed is:

1. A composition comprising an amount of dicalcium phosphate and an amount of an orange peel derivative.

2. The composition of claim 1, wherein the orange peel derivative comprises at least one selected from the group consisting of orange peel powder, orange peel extract, orange peel essential oil, orange peel wax, and any combination thereof.

3. The composition of claim 2, wherein the composition further comprises bee pollen, horsetail herb, cabbage powder, and stevia.

4. The composition of claim 2, wherein the composition is administered to a subject in an amount effective to treat, ameliorate, or reduce the effects of a condition selected from the group consisting of inflammation, pain, a cardiovascular disorder, osteoporosis, a bone injury, low metabolism, anxiety, autism spectrum disorder, a sleep disorder, a neurodegenerative disorder, depression, scoliosis, degenerative disk disease, muscular dystrophy, and any combination thereof.

5. The composition of claim 4, wherein the condition is scoliosis.

6. The composition of claim 2, wherein the composition is administered to a subject in an amount effective to perform a function selected from the group consisting of increasing cellular glucose-6-phosphate production, improving range of motion, improving DNA methylation, increasing folate levels in the brain, improving nerve impulse, improving immune function, improving teeth alignment, increasing the production of collagen, increasing the production of L-carnitine, increasing the production of noradrenaline, improving the regulation of carnitine, improving the regulation of noradrenaline, increasing the release of catecholamine, increasing the release of acetylcholine, and any combination thereof.

7. The composition of claim 1, wherein a ratio of the amount dicalcium phosphate to the amount orange peel derivative is between 10:1 and 1:10 by weight.

8. The composition of claim 7, wherein the ratio of the amount dicalcium phosphate to the amount orange peel derivative is about 1:1 by weight.

9. The composition of claim 1, wherein the composition is formulated in a dose between 10 mg and 1000 mg.

10. The composition of claim 9, wherein the composition is formulated in a dose between 100 mg and 300 mg.

11. The composition of claim 4, wherein the composition is administered to the subject between one time per day and four times per day.

12. The composition of claim 11, wherein the composition is administered to the subject three times per day.

13. The composition of claim 11, wherein the composition is formulated as a toothpaste.

14. The composition of claim 11, wherein the composition is formulated for sublingual administration.

15. A method of treating scoliosis in a subject, the method comprising administering the composition of claim 1 to the subject.

16. The method of claim 15, wherein the composition further comprises bee pollen, horsetail herb, cabbage powder, and stevia.

17. The method of claim 16, wherein a ratio of the amount dicalcium phosphate to the amount orange peel derivative is between 10:1 and 1:10 by weight.

18. The method of claim 16, wherein the composition is administered to the subject between one time per day and four times per day.

19. The method of claim 16, wherein the composition is formulated as a toothpaste.

20. The method of claim 16, wherein the composition is formulated for sublingual administration.
